(19) United States
(12) Patent Application Publication
(10) Pub. No.: US 2011/0124035 A1
(43) Pub. Date: May 26, 2011

(54) DEVICE FOR EXPOSING A SENSOR TO A CELL CULTURE POPULATION IN A BIOREACTOR VESSEL

(75) Inventors: Scott T. Broadley, Los Angeles, CA (US); Robert J. Garrahy, Villa Park, CA (US)

(73) Assignee: Broadley-James Corporation, Irvine, CA (US)

(21) Appl. No.: 13/020,726

(22) Filed: Feb. 3, 2011

Related U.S. Application Data
(63) Continuation of application No. PCT/US2009/053215, filed on Aug. 7, 2009.
(60) Provisional application No. 61/087,579, filed on Aug. 8, 2008.

(51) Int. Cl. C12Q 1/02; C12M 1/34

(52) U.S. Cl. 435/29; 435/288.7

(57) ABSTRACT

Devices and methods for exposing a sensor to a cell culture or microbial population are disclosed. In one embodiment, a sensor well for use with a bioreactor vessel includes a sheath; a sensing element disposed on or in a portion of the sheath; a signal transmitter disposed within at least a portion of the sheath and configured to provide signals to and/or receive signals from the sensing element and provide signals to and/or receive signals from a sensor controller; a connector configured to attach the sensor well to a portion of a bioreactor vessel, the connector including an aperture through which the sheath can be deployed into the bioreactor vessel; and a collapsible bellows which houses the sheath when in an undeployed position, the bellows coupled to one end of the sheath, the bellows, the connector, and the sheath configured to form at least a portion of a hermetically sealable and sterilizable enclosure.

### RELATED APPLICATIONS

This application is a continuation of International Application No. PCT/US2009/053215, filed Aug. 7, 2009, which claims the benefit of U.S. Provisional Application No. 61/087,579, filed Aug. 8, 2008. The disclosures of all of the above-referenced prior applications, publications, and patents are considered part of the disclosure of this application, and are incorporated by reference herein.

### BACKGROUND OF THE INVENTION

1. Field of the Invention

This application, generally relates to devices and methods for use with disposable bioreactor systems. More particularly, this application relates to disposable probes for exposing a cell culture population to fluorescence dots.

2. Description of the Related Art

Bioreactor systems (“bioreactors”) or fermenters include containers which are used for fermentation, enzymatic reactions, cell culture, tissue engineering, and food production, as well as containers used in the manufacture of biologicals, chemicals, biopharmaceuticals, microorganisms, plant metabolites, and the like. Bioreactors vary in size from benchtop systems to large stand-alone units. The containers or “vessels” used in bioreactor systems can vary in size from less than about one (1) liter to about one thousand (1000) liters or more. The stringent asepsis requirements for sterile production in some bioreactors can require elaborate systems to achieve the desired product volumes. Consequently, the production of products in aseptic bioreactors can be costly which provides the motivation for pursuing improved systems. Pre-sterilized disposable bioreactor systems or “bioreactor bags” have been developed that need not be cleaned, sterilized or validated by end users, all of which can lower production costs.

Current practice for monitoring cell culture populations in standard glass and steel bioreactors involves introducing applicable probes, such as temperature, pH, or dissolved oxygen or dissolved carbon dioxide probes, through a port in the reactor wall or head plate. Some systems incorporate optical-based probes and other devices for measuring dissolved oxygen, pH, and dissolved CO₂. However, current probes and devices do not address significant issues related to sterilization requirements, sensor disposability and cost, and sensor shelf life which make the current devices less than optimal, especially when used in conjunction with a disposable bioreactor bag.

### SUMMARY OF CERTAIN EMBODIMENTS

The system, method, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of Certain Embodiments” one will understand how the features of this invention provide advantages over other sensors.

Embodiments of a sensor well that can be used with a bioreactor vessel, including a disposable bag, are illustrated and described herein. In one embodiment a sensor well for use with a bioreactor vessel includes a sheath; a sensing element disposed on or in a portion of the sheath; a signal transmitter (e.g., a waveguide) disposed within at least a portion of the sheath and configured to provide signals to and/or receive signals from the sensing element and provide signals to and/or receive signals from a sensor controller; a connector configured to attach the sensor well to a portion of a bioreactor vessel, the connector comprising an aperture through which the sheath can be deployed into the bioreactor vessel; and a collapsible bellows which houses the sheath when in an undeployed position, the bellows coupled to one end of the sheath, the bellows, the connector, and the sheath configured to form at least a portion of a hermetically sealable and sterilizable enclosure, wherein deploying the sheath through the aperture in the connector exposes the sensing element is exposed to media contained in a bioreactor vessel while maintaining a sterile environment in the bioreactor vessel. The sensing element can comprise fluorescing material and may be configured as a fluorescent dot. The sensor well can also include a plurality of sensing elements disposed on the sheath and a plurality of signal transmitters disposed at least partially within the sheath, each signal transmitter associated with a least one sensing element. In one embodiment, the sensing element includes an electronic sensor. In another embodiment, the signal transmitter includes an optical waveguide. The bellows can comprise a telescoping structure or a flexible material configured in an accordion-like structure. In some embodiments, the fluorescing material can be disposed on, or impregnated in, a sheath, for example a plastic sheath. In some embodiments, the fluorescing material is affixed to the sheath by a translucent material, having adhesive properties, such that the sensor is exposed to the media when deployed. The fluorescing material can be configured to indicate a characteristic or measurement of the cell culture to which it is exposed, including for example, pH, dissolved oxygen, or carbon dioxide. The sensing element can also be an electronic sensor which can be configured to sense, for example, temperature, conductivity, or osmolality. The signal transmitter can include an optical waveguide, wires, or other means to convey a signal from and/or to the sensing element.

A device for use with a bioreactor system is also provided. The device includes a waveguide; a sheath surrounding the waveguide; a sensing element disposed at a distal end of the sheath, the sheath and the sensing element configured to move between a first position and a second position; a sterilizable enclosure configured to protect the sensing element from an exterior environment at least when the sensing element and the sheath are in the first position; and a sterile connector disposed at a distal end of the enclosure, wherein the distal end of the sheath is disposed distal of the sterile connector when the sensing element and the sheath are in the second position. In one embodiment, the sensing element includes a fluorescing material. In another embodiment, the sensing element includes one or more dots, each comprising fluorescing material. In some aspects, at least one of the dots is configured to fluoresce, when radiated, to indicate a characteristic of a cell culture population to which it is exposed. The one or more dots can be configured to fluoresce to indicate pH, dissolved oxygen content, or carbon dioxide content. In one embodiment, the sensing element is an electronic sensor. The sensing element can be configured to measure temperature, conductivity, or osmolality.

Also provided is a device for use with a bioreactor vessel. The device includes a sensor comprising a signal transmitter, a sheath, the sheath at least partially surrounding the signal transmitter, and at least one sensing element disposed at a distal end of the sheath, the sensor configured to move between a first and a second position; bellows surrounding at least a portion of the sensor at least when the sensor is in the first position; and a sterile connector disposed at a distal end of the bellows, wherein the bellows, the sterile connector, and the sheath form a sterilizable enclosure when the sensor is in the first position, and wherein the sensor is movable with respect to the connector such that the distal end of the sheath is disposed distal of the sterile connector when the sensor is in the second position. In one embodiment, the signal transmitter includes an optical waveguide and the sensing element includes a fluorescent dot. In another embodiment, the signal transmitter includes a communication wire.

In another embodiment, a device for use with a bioreactor vessel includes a sensor comprising at least one signal transmitter and at least one sensing element; and an enclosure defining a sterilizable space around the sensor, the enclosure comprising a connector configured to provide a sterile connection between the enclosure and a corresponding connector on a bioreactor vessel.

In yet another embodiment a device for monitoring media in a bioreactor bag having at least one port with a first sterile connector coupled thereto is provided. The device includes means for monitoring a characteristic of the media, the monitoring means comprising at least one fluorescent dot, means for transmitting light to the fluorescent dot, and means for transmitting a response signal from the fluorescent dot to a controller; means for protecting a sterile environment of the monitoring means; means for making a sterile connection between the monitoring means and the first sterile connector; and means for inserting the monitoring means into the bioreactor bag once a sterile connection has been made. In one embodiment, the means for transmitting light includes an optical waveguide. In another embodiment, the means for transmitting a response signal from the fluorescent dot to the controller comprises an optical waveguide.

Another embodiment includes a bioreactor system. The bioreactor system includes a disposable bioreactor bag comprising at least one port having a first sterile connector coupled thereto; a sensor comprising at least one signal transmitter, at least one sensing element, and a second sterile connector; and an enclosure defining a sterilizable space around at least the sensing element, the enclosure comprising a connector configured to provide a sterile connection between the enclosure and a corresponding connector on a bioreactor bag.

Another embodiment includes a method of monitoring media in a bioreactor vessel having at least one port with a first sterile connector coupled thereto. The method includes coupling the first sterile connector of the bioreactor vessel to a disposable sensor well, the sensor well including bellows, an injectable member, and a second sterile connector having a seal, the bellows, the injectable member, and the second sterile connector forming a sterilizable space therebetween, the injectable member comprising at least one waveguide and at least one fluorescent dot, the fluorescent dot being disposed in the sterilizable space before the injectable member is injected into the bioreactor vessel; removing the seal of the second sterile connector; and injecting the injectable member into the bioreactor vessel.

### BRIEF DESCRIPTION OF THE DRAWINGS

The above mentioned and other features of the invention will now be described with reference to the drawings of a preferred embodiment of the present sensor well. The illustrated embodiment is intended to illustrate and not to limit the invention. The drawings contain the following figures:

FIG. 1 is a cross sectional view of a sensor well according to an embodiment of the invention, shown in an undeployed position.

FIG. 2 is a cross-sectional view of a sensor well according to another embodiment, shown in an undeployed position and coupled to a bioreactor vessel.

FIG. 3 is a cross-sectional view of the sensor well in a deployed state.

FIG. 4 is a perspective view of the sensor well of FIG. 2, shown housed in a sterile package.

FIG. 5 a perspective view of the sensor well of FIG. 4, shown removed from the package and with its sealing layer removed.

FIGS. 6A through 6E show cutaway perspective views of sensor wells configured in accordance with various embodiments.

FIG. 7 is a schematic drawing illustrating a portion of an exemplary bioreactor system can be used with embodiments of the invention.

FIG. 8 is a schematic drawing illustrating a sensor well embodiment in use with the bioreactor system of FIG. 7.

FIG. 9a is a side view of one embodiment of a sensor well in an undeployed state.

FIG. 9b is a cross-sectional view of the sensor well of FIG. 9a in an undeployed state, with the signal transmitter inserted into the sheath.

FIG. 9c is a cross-sectional view of the sensor well of FIG. 9a in an undeployed state, with the signal transmitter removed from the sheath.

FIG. 10a is a side view of another embodiment of a sensor well in an undeployed state.

FIG. 10b is a cross-sectional view of the sensor well of FIG. 10a in an undeployed state, with the signal transmitter inserted into the sheath.

FIG. 10c is a cross-sectional view of the sensor well of FIG. 10a in an undeployed state, with the signal transmitter removed from the sheath.

FIG. 11 is a cross-sectional view of an embodiment of the sensor well that includes inlet and outlet ports.

### DETAILED DESCRIPTION OF CERTAIN EMBODIMENTS

Reference will now be made in detail to the preferred embodiments of the invention, examples of which are illustrated in the accompanying drawings. While the invention will be described in conjunction with certain preferred embodiments illustrated and described herein, it will be understood that they are not intended to limit the invention to those embodiments. On the contrary, the invention is intended to cover alternatives, modifications and equivalents, which may be included within the spirit and scope of the invention as defined by the appended claims. In addition, features of the invention that are described in one embodiment are not limited to that embodiment unless specifically stated as such; instead, certain features may be suitably used in the other embodiments described herein or other embodiments of the invention that are not specifically described.

Some existing optical sensing systems designed for use with disposable bioreactor bags may include sensing material, for example, fluorescing material, which is incorporated into the design of the bioreactor bag. Such designs present a number of disadvantages. For example, to meet sterilization requirements, some sensors are incorporated into the bag and sterilized with the bag, and then supplied to a customer as a bioreactor bag having a certain sensor. In preferred embodiments, the sensor well comprises materials that can be gamma ray sterilized. This can present logistics problems as well as increase costs, particularly for optical-based sensors which cannot be steam sterilized and must be irradiated instead. Also, bioreactor bags have a certain shelf life (for example, about eighteen (18) months). Fluorescence material used in many optical sensors also has a shelf life that is typically shorter than the bioreactor bags (for example, about one year). Accordingly, the expiration of fluorescence material incorporated into a disposable bioreactor bag can shorten the shelf life of the bioreactor bag. In addition, certain calibration information is associated with the fluorescence material. The calibration information must be available when using the bag to ensure correct readings of the fluorescence material. Accordingly, a sensor containing fluorescence material incorporated into the bag requires that the sensor calibration data always “travel” with the bag, which can increases logistics problems and expenses.

Embodiments of the invention desirably provide a sensor well for use with a bioreactor vessel or container. The sensor well is configured to maintain a sterile environment around sensing components which can include one or more optical or electronic sensors. The sensor well is configured such that it can be connected, in a sterile manner, to a sterile bioreactor vessel before or after the vessel is provided from the vessel manufacturer and still maintain the sterile environment inside the vessel. This is one of the advantages of this sensor well. This allows, for example, a user to select a particular bioreactor bag from stock and then configure the bag with a particular sensor well, if desired. In other words, the sensor well can be provided to a customer packaged and sterilized, and the customer can store the sensor well and introduce it into a bioreactor vessel when desired or deemed necessary. Calibration data relating to the sensor well can be packaged and stored with the sensor well so that it is readily available when the sensor well is installed into a bioreactor vessel. The sensor well configuration allows it to be connected while the vessel is in use so that a sensor can be deployed in the bioreactor vessel and still maintain a sterile environment inside the vessel, so long as the bioreactor vessel is suitably configured with a connector for mating with a matching connector of the sensor well. The embodiments of the sensor well generally described herein are described for use with a disposable bioreactor bag, but the embodiments should not be construed as limited to such use. Instead, embodiments of the sensor well can be equally used with a hard-walled bioreactor vessel, for example, one made from steel or glass.

Specifically, embodiments illustrated and described herein provide a sensor well which can be introduced into a sterile environment for example in a single-use disposable vessel of a bioreactor system or another type of bioreactor system. Various embodiments can be configured with one or more sensing element so as to sense one or more desired characteristics of media in a bioreactor vessel. The sensor well can include one or more of many types of sensors, including but not limited to optical sensors including fluorescing sensors, potentiometric ion-selective sensors, amperometric sensors, and resistive sensors. In addition, embodiments advantageously provide a sensor whose calibration data remains with the sensor well, separate from the bioreactor bag or other vessel with which the sensor is used, so the particular sensor well can be calibrated into any vessel the sensor is placed in when it is actually placed in operation.

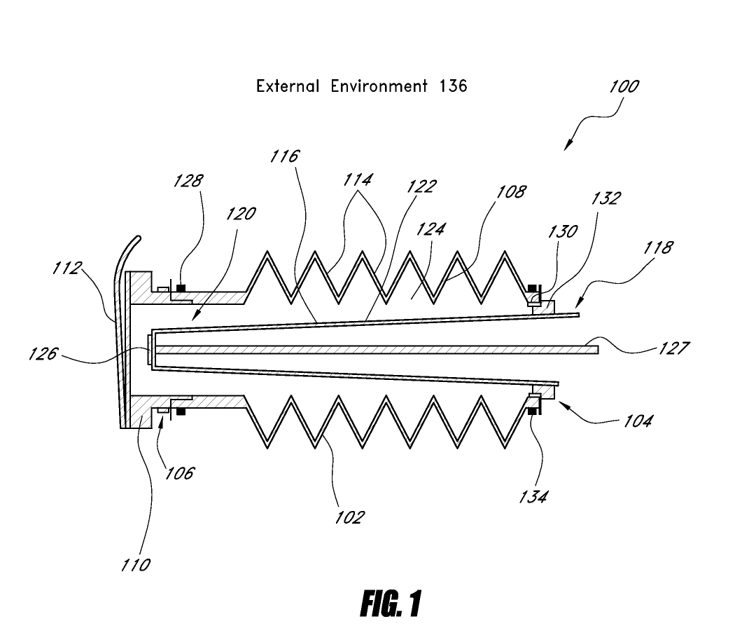

FIG. 1 illustrates an embodiment of a sensor well 100 shown in FIG. 1 in an undeployed state. The sensor well 100 includes an enclosure 102 having a proximal end 104, a distal end 106, and an inner surface 108. A connector 110 is disposed at the distal end 106 of the enclosure 102. A removable sealing layer 112 covers the distal end 106 of the enclosure 102 and hermetically seals the enclosure 102. The enclosure 102 also includes one or more bellows 114 disposed between the proximal end 104 and the distal end 106. The bellows 114 are configured to be compressible, such that the proximal end 104 of the enclosure 102 can be moved closer to the distal end 106 of the enclosure 102.

The sensor well 100 also includes a sheath or injectable member 116 disposed substantially within the enclosure 102. In FIG. 1 the sensor well 100 is in an undeployed state such that the sheath is still within the enclosure 102. The sheath 116 has a proximal end 118, a distal end 120, and an outer surface 122. Together, the inner surface 108 of the enclosure 102 and the outer surface 122 of the sheath 116 form a sterilizable space 124 within the enclosure 102. A sensing element 126 is disposed at the distal end 120 of the sheath 116, inside the enclosure 102. The sensing element 126 communicates with a signal transmitter 127 disposed inside the sheath 116, outside of the sterilizable space 124. The sheath 116, sensing element 126 and the signal transmitter 127 are sometimes collectively referred to herein as the “sensor.” The sheath 116 is coupled to the proximal end 104 of the enclosure 102 such that the sheath 116 can move with the proximal end 104 when the proximal end 104 is moved toward the distal end 106 of the enclosure 102. By such a configuration, the well 100 can be provided to a user in an undeployed and presterilized state, for example, inside a hermetically sealed plastic bag with the sealing layer 112 sealing the opening in the connector 110. The user can then couple the well 100 to a suitable connector, such as a sterile connector disposed on a bioreactor bag, remove the sealing layer 112 and an associated sealing layer on the connector disposed on the bioreactor bag, and deploy the sheath 116 into the bioreactor bag without exposing the sensing element 126 or the interior of the bioreactor bag to an unsterile environment. In addition, the user can replace the signal transmitter 127 without necessarily removing the well 100 from the bag because the proximal end 118 of the sheath may be open or exposed to the environment outside the bag allowing access to components in the sheath.

In the embodiment illustrated in FIG. 1, the connector 110 is attached to the enclosure 102 and forms a part of the enclosure 102. The connector 110 can be held in place by a clamp (or securing band) 128 which at least partially surrounds the joint between the connector 110 and the enclosure 102. In this embodiment, a threaded insert 130 and a locking nut 132 cooperate to secure the sheath 116 at the proximal end 104 of the enclosure 102. An additional clamp 134 can optionally be included to surround the joint between the sheath 116 and the proximal end 104 of the enclosure 102 and provide additional securement. Although not illustrated, one or more o-rings or other types of seals can be included at this joint to provide an airtight seal at this joint. Of course, any suitable means can be used to couple the sheath 116 to the enclosure 102, so long as the means provides an airtight seal between the interior 124 of the sensor well 100 and the exterior environment 136.

In some embodiments, the enclosure 102 can comprise a syringe plunger sheath, such as the Silicone Syringe Plunger Sheath which can be purchased from Qosina Corporation of Edgewood, N.Y. The illustrated embodiments show an enclosure 102 having bellows 114 having an accordion-like structure which is configured to compress and allow the sheath 116 to move from a first undeployed position to a second deployed position while maintaining a sterile environment inside the enclosure 102. However, other embodiments of the invention can have any other suitable configuration of material to achieve this functionality while maintaining a sterile interior in the enclosure 102. For example, some embodiments can include enclosures having a telescoping configuration. Other examples of bellows include other flexible material(s) that allow the sensor well to be adequately deployed into a bioreactor vessel.

In some embodiments, the connector 110 can be a Kleenpak™ Connector available from PALL Corporation of East Hills, N.Y. Of course, any other suitable connector configured to sterilely connect two fluid environments can also be used with embodiments of the invention.

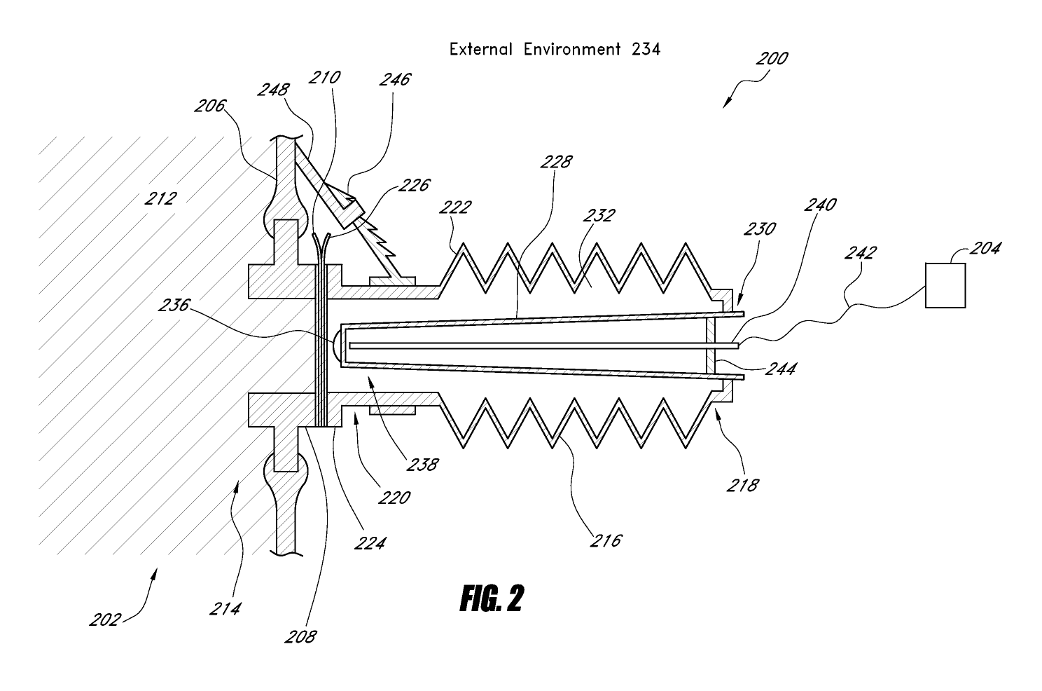

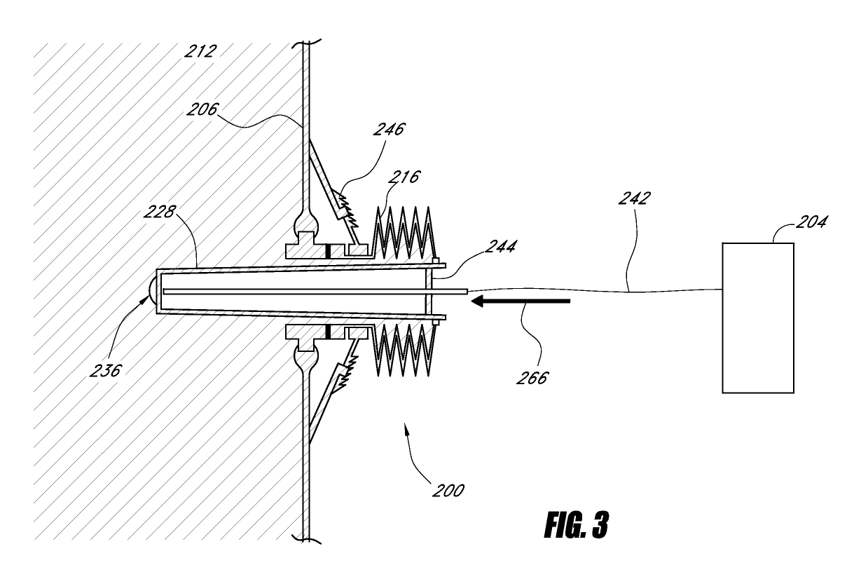

FIGS. 2 and 3 illustrate the deployment of a sensor well 200 configured according to one embodiment. FIG. 2 shows the sensor well 200 in an undeployed state, coupled to a bioreactor system comprising a vessel 202 and a sensor controller (or processor) 204 disposed outside of the vessel 202 and as part of a bioreactor control system. In this embodiment the bioreactor control system includes a flexible bioreactor bag 206, having at least one sterile connector 208 disposed on the bag 206. The connector 208 is covered by a removable sealing layer 210. A sterile environment is maintained in the bioreactor bag 206, which when operable may be at least partially filled with media 212, such as a cell culture population. The connector 208 is configured to allow the interior 214 of the bioreactor bag 206 to be fluidly connected to a second sterile environment, such as, for example, a fluid supply line, without exposing either the interior 214 of the bioreactor bag 206 or the second sterile environment to an unsterile environment.

The sensor well 200 includes an enclosure 216 having a proximal end 218, a distal end 220, and compressible bellows 222 disposed therebetween. A connector 224 is disposed at the distal end 220 of the enclosure 216, and is covered by a sealing layer 226. Disposed substantially within the enclosure 216 is a sheath 228. The sheath 228 has a proximal end 230 which is sealingly coupled to the proximal end 218 of the enclosure 216 so as to separate an interior environment 232 of the enclosure 216 from the ambient, or external environment 234. The sheath 228 is configured to move in a longitudinal direction as the bellows 222 are compressed (that is, as the proximal end 218 of the enclosure 216 is moved towards the distal end 220 of the enclosure 216). For example, as illustrated in FIG. 2 the sensor well 200 is configured such that the sheath 228 moves horizontally to the left when deployed into a bioreactor vessel and moved from a first undeployed position to a second deployed position. A sensing element 236 is disposed at a distal end 238 of the sheath 228, within the interior environment 232 of the enclosure 216. In the illustrated embodiment, the sensing element 236 comprises fluorescent material (for example, a fluorescent dot). The sensing element 236 can be configured to provide an indication of a characteristic of the media it is exposed to, for example, pH, dissolved oxygen, or dissolved carbon dioxide.

A fluorescent “dot” or “patch” is typically a section of polymer sheet that has been coated or impregnated with a fluorescent compound or mixture of compounds that fluoresce when excited by a proper light source. When excited by a light source with proper wavelength, the fluorescent compound fluoresces and then if the excited fluorescent compound encounters an analyte, the analyte affects (e.g., changes or quenches) the fluorescent signal. The fluorescence intensity or phase shift (decay time, preferred for reasons of system stability, precision and accuracy) measured by a spectrometer system is related to the analyte concentration.

Dot/patch sizes typically vary from a few millimeters to several centimeters in diameter. Thickness can vary from less than a quarter of a millimeter to several millimeters. The dot/patch can be covered with a layer of material to trap the fluorescent compound in the matrix of the dot/patch to prevent the fluorescent compound (for example, ruthenium complex or porphyrin) from leaching into the sample. An optical transparent adhesive can be used to attach the dot to different surfaces. The fluorescent compound can also be directly painted/applied on a supporting substrate when the there is no concern of leaching (such as when the fluorescent compound is chemically linked to the supporting substrate matrix).

Thus, fluorescent dots (“dots”) as used herein, is a broad term that refers to a mass of material that is configured to have certain fluorescing characteristics (for example, to have a particular fluorescence to indicate pH, dissolved oxygen content, or CO₂ content), and that is disposed on a distal end of the sheath. In various applications, dots may be configured in various sizes (e.g., length, width, and height), shapes, colors, and compositions to exhibit desired sensing characteristics. Some examples of dot cross-sectional shapes includes circles, ovals, generally curvilinear shapes, squares, rectangles, triangles, generally polygonal shapes, and irregular shapes. In a configuration that includes numerous dots, the dots may be disposed in a uniform pattern or another pattern to produce a desired sensing effect, and each dot may each be about the same size or the dots can vary in size. A fluorescent dot can be three-dimensional or essentially two-dimensional, with very little or negligible thickness extending along a longitudinal axis of the sheath. In one embodiment, the thickness of the fluorescent dot is less than the width or length of the dot. In another embodiment, the dot has a relatively low profile at the distal end of the sheath.

A signal transmitter 240 is disposed at least partially within the sheath 228, separated from the interior environment 232 of the enclosure 216 by the sheath 228. The signal transmitter 240 is configured to transmit a signal to and/or from the sensing element 236 to the processor 204 via a connection line 242. In the illustrated embodiment, the signal transmitter 240 is an optical waveguide, configured to transmit a particular wavelength of light to illuminate the sensing element 236. The signal transmitter 240 is configured to transmit a return signal from the sensing element 236 back to the processor 204. A characteristic of the fluorescing material can be used to indicate a property of characteristic of media. For example, the wavelength or frequency of fluorescence, or the rate at which the fluorescence of the material decays can be used to determine a characteristic property of the bioreactor media 212, such as, for example, pH, dissolved oxygen, or other characteristics of the media 212. The distal end 238 of the sheath 228 can have a transparent window (not visible in FIG. 2) so as to allow a signal to be transmitted to and from the sensing element 236 through the window. The signal transmitter 240 is held in place by a support, or sensor holder 244 disposed within the sheath 228. In some embodiments, the signal transmitter 240 can be substantially disposed inside a sensor component enclosure within the sheath 228.

As shown in FIG. 2, the connector 224 of the sensor well 200 is configured to mate with the connector 208 of the bag 206. The sensor well 200 and/or the bag 206 can include one or more features configured to aid in securement of the well 200 to the bag 206 when the connectors 224, 208 are coupled together. For example, the sensor well 200 can include a tether, or sensor well support, such as the illustrated ratcheted tether 246, coupled to the distal end 220 of the well 200. The tether 246 is configured to cooperate with a corresponding support 248 on the bioreactor bag 206 to support the well 200 when it is connected to the bag 206.

FIG. 2 illustrates the sensor well 200 connected to the bioreactor bag 206 with the sealing layers 210, 226 intact and the sensor well in an undeployed position. In such a position, the distal end 238 of the sheath 228 is disposed proximal of the distal end 220 of the enclosure 216, within the interior environment 232 of the well 200. Once the sensor well 200 is connected to the bag 206, the sealing layers 210, 226 can be removed to expose the interior 214 of the bag 206 to the interior environment 232 of the enclosure 216. In some configurations the sealing layers 210, 226 are removed by pulling them together in a direction about orthogonal to the attached sensor well 200. The proximal end 218 of the enclosure 216 can then be pushed toward the distal end 220 of the enclosure 216, thereby compressing the bellows 222 and moving the distal end 238 of the sheath 228 past the connector 224 and into the bag 206.

FIG. 3 illustrates the sensor well 200 in this deployed position inside the bag 206. To deploy the sensor well 200, a force is generally applied in the direction indicated by an arrow 266. In the deployed position, the sensing element 236 is exposed to such the media, or bioreactor contents 212 inside the bioreactor bag 206 so that it may perform its sensing function. For example, the sensing element 236 can be placed in contact with, in proximity to, or placed in the same environment as the media such that the media affects the sensing element 236. The aseptic condition within the bioreactor 206 is maintained, however, as the only exposure caused by insertion of the well 200 into the bag 206 is exposure to the pre-sterilized enclosure 216, the sheath 228, and the sensing element 236. Although not illustrated, the sensor well 200 and/or the bag 206 can be provided with one or more features to releasably or fixedly secure the well 200 in the deployed position. Such features can include any suitable means for achieving this function, including one or more tethers, clamps, or latches.

FIG. 4 shows a perspective view of the sensor well 200 as it can be provided to the user, pre-sterilized and sealed within a sterile package 250. The sensor well 200 can be delivered with seal 226 in place such that the sensor well 200 is ready for use upon opening the package. Embodiments of the invention can be sold, transported, and stored in the sterile package 250, and, thus, the sterility of the sensor well 200 can be maintained until it is needed. In embodiments in which the sensor element (not shown) comprises a light-sensitive element such as a fluorescent dot, the sterile package 250 can be opaque so as to protect the sensor element from degradation to ambient radiation. Additionally, the configuration of the enclosure 216, in cooperation with the sealing layer 226 of the connector 224, maintain the sterility of the internal environment of the enclosure 216 until such time as the sensor well 200 is coupled to a mating connector on a bioreactor vessel.

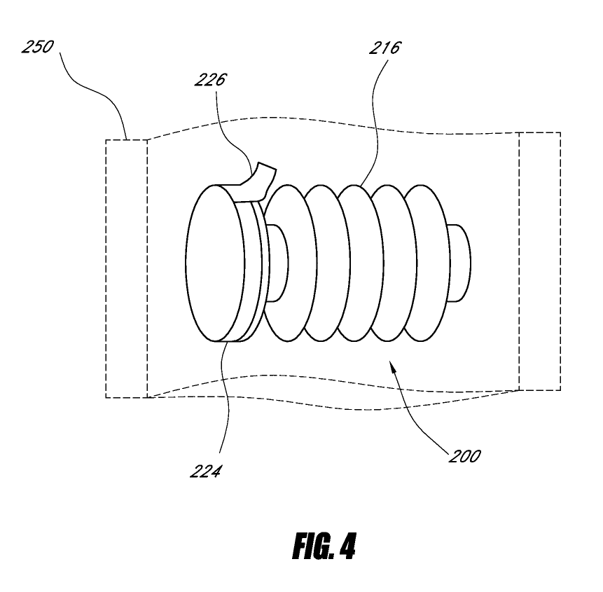

FIG. 5 shows a perspective view of the sensor well 200 removed from the package 250. Here, the sealing layer 226 is shown removed to better illustrate the sensing element 236, parts of the sheath 228, and the interior 252 of the enclosure 216.

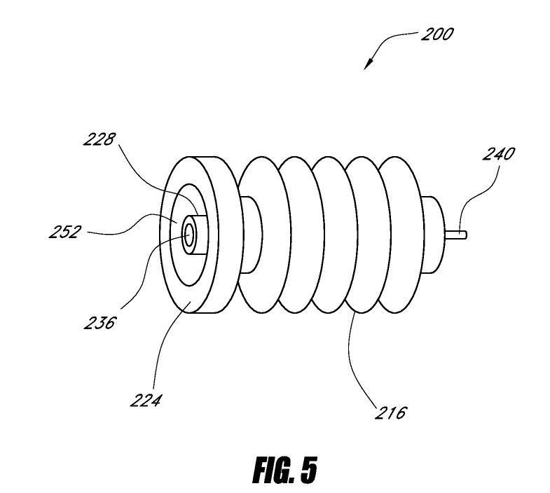

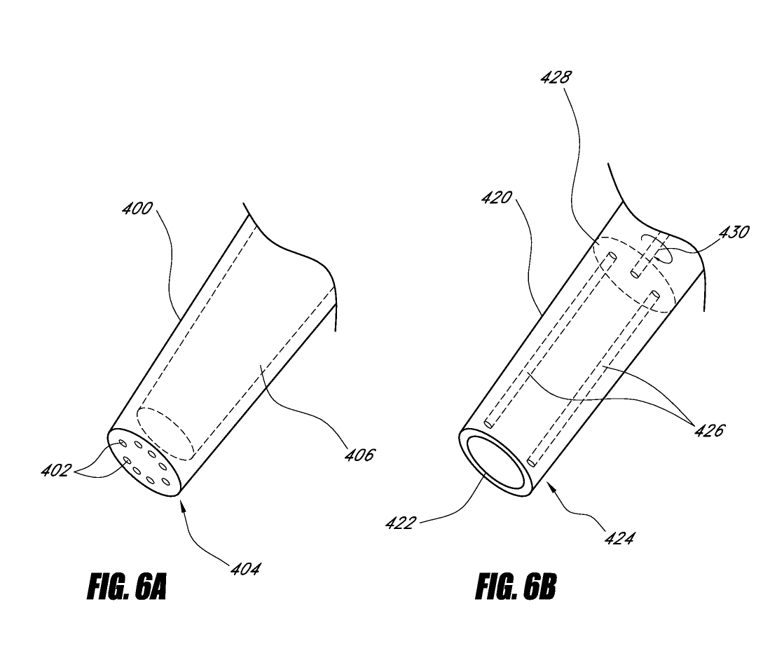

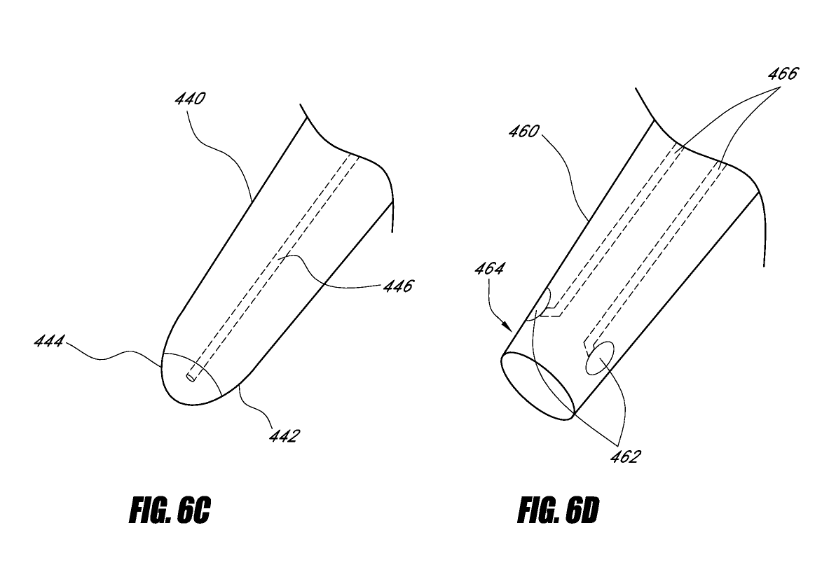

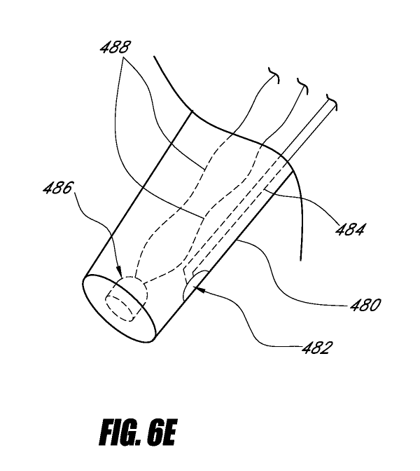

With reference now to FIGS. 6A through 6E, various configurations of sensors and sheaths are illustrated in accordance with embodiments of the invention. FIG. 6A shows a sheath 400 having a plurality of sensing elements 402 disposed at a distal end 404 thereof. The sensing elements 402 can comprise fluorescent material configured as dots disposed in a spaced-apart fashion on the exterior surface of the distal end 404 of the sheath 400. Inside the sheath 400, and shown in dashed lines, is a signal transmitter 406, which may be an optical waveguide configured to transmit light both to and from the sensing elements 402. Although not illustrated, the distal end 404 of the sheath 400 can include a transparent window on which the sensing elements 402 are disposed to allow light to pass to and from the sensing elements 402.

Thus, some embodiments comprise fluorescent dots disposed on the exterior surface of the distal end 404 of the sheath 400. As described in greater detail above, when the fluorescent compound is excited by a light source with proper wavelength, the fluorescent dot fluoresces. When the sheath 400 is deployed inside the bioreactor bag, the fluorescent dot is placed in contact with the media, or bioreactor contents, inside the bioreactor bag so that it may perform its sensing function. The bioreactor contents held inside the bag, for example a cell culture population, may contain an analyte. If the excited fluorescent compound encounters analyte when the sheath 400 is deployed inside the bioreactor bag, the analyte quenches the fluorescent signal. The fluorescence intensity or phase shift measured by a spectrometer system can then be related to the analyte concentration.

In some embodiments, multiple signal transmitters may be included in the sheath 400. In such embodiments, each signal transmitter may be associated with one or more of the fluorescing dots. As shown in the FIG. 6A, in this embodiment the signal transmitter 406 is dimensioned such that a surface of the signal transmitter 406 can receive a signal from all of the sensing elements 402. As will be appreciated by one skilled in the art, by using a plurality of sensing elements, a sensor well can be configured to detect several different properties, or several different ranges of the same property.

FIG. 6B illustrates another exemplary embodiment of a sheath 420. In this embodiment, sheath 420 comprises a single deposit of fluorescent material 422 which is disposed on the distal end 424 of the sheath 420. The fluorescent material 422 substantially covers the distal end 424. Disposed inside the sheath 420 are multiple waveguides, this particular example includes two relatively small waveguides 426. The waveguides 426 are mounted on a disk 428 which, in turn, is connected to a shaft 430. This system is configured such that rotation of the shaft 430 causes the waveguides 426 to rotate about the axis of the shaft 430, exposing different areas of the fluorescent material 422 to each of the waveguides 426 at different times. The shaft 430 can be configured to rotate, translate, or achieve a combination of these motions. In addition, the shaft 430 can be configured to move continuously, intermittently at a predetermined rate, or when desired by a user. With such an embodiment, the user is able to move the waveguides to a different area of the fluorescent material 422 as the previously used portion deteriorates, without needing to recalibrate test equipment.

FIG. 6C illustrates one of several tip geometries that may be incorporated with embodiments of the invention. A sheath 440 includes a generally bullet-shaped distal tip 442, upon which a sensing element 444 is disposed. A signal transmitter 446 is disposed inside the sheath 440, and is configured to communicate a signal, such as an optical signal, to and from the sensing element 444. Of course, as will be apparent to one skilled in the art, embodiments of the present invention encompass a variety of alternative tip geometries in addition to the ones illustrated.

FIG. 6D is an illustration of an embodiment comprising a sheath 460 having one or more sensing elements 462 located on a side of the sheath 460, toward a distal end 464 of the sheath 460. Such an embodiment can include one or more signal transmitters 466 disposed inside the sheath 460. The signal transmitters 466 can extend substantially parallel to the longitudinal axis of the sheath 460 toward the distal tip 464, and can curve or angle as they approach the distal tip so as to communicate with the sensing elements 462. Embodiments can also include sensing elements disposed on the side of the sheath 460 and/or on the end of the sheath 460 even closer to the proximal end of the sheath.

FIG. 6E illustrates a sheath 480 according to a further embodiment of the invention. The sheath 480 includes multiple types of sensors, including an optical sensor comprising a fluorescing material 482 disposed on a side of the sheath 480, and a signal transmitter 484 configured to communicate with the dot 482. The sheath 480 also includes a second sensor comprising a sensing element 486 and a communication line, or wire 488. It will be apparent to one skilled in the art that numerous embodiments of the invention can include multiple and diverse sensors in a single sheath.

Although the illustrated embodiments generally include an optical sensor comprising fluorescent material and a waveguide signal transmitter, alternative embodiments can include any other type of sensor, such as a pH electrode or any other conventional pH or dissolved oxygen sensor, a thermal well, sensors configured to sense conductivity or osmolality, or any other type of sensor which is desirably placed in direct contact with media. Embodiments of the invention can also include any desired combination of sensors.

Persons of skill in the art will also understand bioreactor systems described herein can include multiple and diverse signal transmitters. For example, signal transmitters can include, but are not limited to, optical waveguides, communication lines or wires, and other means to convey a signal from and/or to the sensing element.

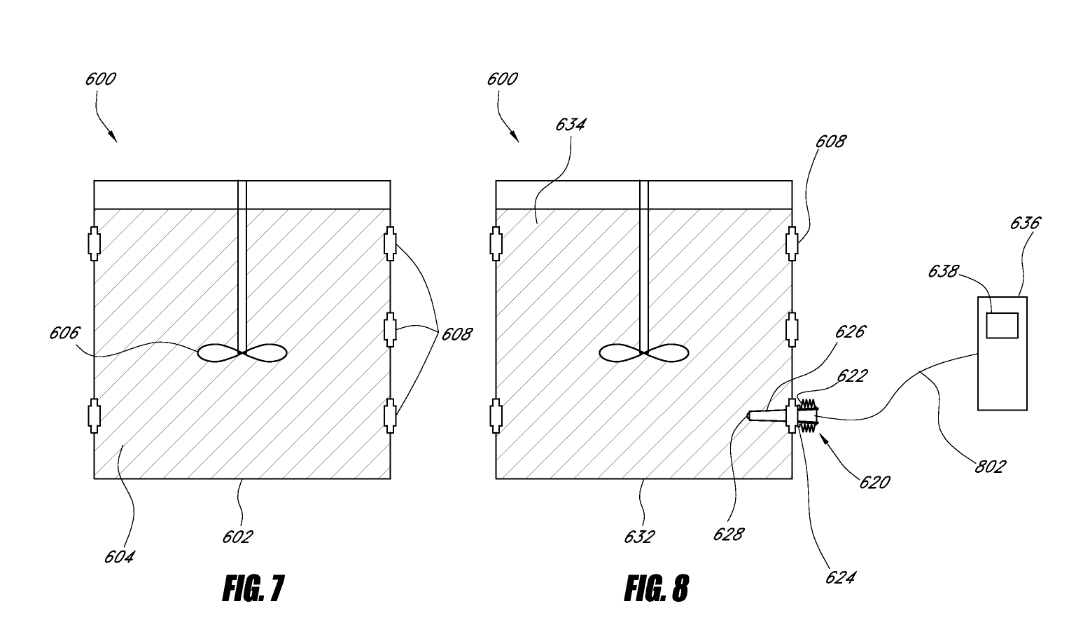

With reference now to FIG. 7, an exemplary bioreactor system 600 is illustrated with which the invention may be used. The system 600 comprises a bioreactor vessel 602, which can be configured to be re-usable (for example, comprising metal or glass) or disposable. The vessel 602 is configured to maintain a sterile environment inside the vessel 602. In operation, the vessel 602 can be at least partially filled with media 604. The vessel 602 includes an agitator 606 configured to agitate the media 604. In this embodiment the agitator 606 comprises a mechanical revolving structure but other agitators can also be used. The vessel 602 also includes one or more sterile connectors, or sterile ports 608 configured to allow fluid communication between the interior of the vessel 602 and a second fluid environment.

FIG. 8 illustrates an embodiment of a sensor well 620 deployed in a media filled vessel 632 of the bioreactor system 600. The sensor well 620 includes an enclosure 622, a connector 624, and a sheath 626 with a sensing element 628 disposed at its distal end. The sensing element 628 provides a signal which is communicated to a processor 636 using a signal transmitter 802 which comprises a portion disposed in the sheath 626 and a portion outside of the sheath 626 which is connected to the processor 636. The sensor well 620 is shown mated with one of the connectors 608 of the bioreactor vessel 632, with the sheath 626 deployed into the interior of the vessel 632 so that the sensing element 628 is exposed to the media 634. The signal transmitter 802 is in communication with processor 636, which is configured to receive and interpret the signal from the sensing element 628. The processor 636 can include a display portion 638 to display the information indicative of a characteristic or property of the media 634 sensed by the sensing element 628.

To deploy the sensor well 620 in the bioreactor system 600, a user can first select the appropriate pre-sterilized sensor well 620 for the particular application. The sensor well 620 can be removed from its packaging, and positioned such that the connector 624 is mated with the corresponding connector 608 of the bioreactor system 600. Once the two connectors 624, 608 are mated, any sealing layers provided on the connectors 624, 608 can be removed, preferably simultaneously, to expose the pre-sterilized interior of the sensor well 620 to the aseptic interior of the bioreactor system 600. Then, the proximal end of the sensor well 620 can be pressed toward the connectors 624, 608 so as to insert the sensor well's sheath 626 into the interior of the bioreactor system 600. Once deployed, the sensing element 628 is exposed to the media 634. The sensor well 620 can optionally be locked into this deployed position, such that the sheath 626 is held in place contacting the media 634. The state of the media 634 can thus be monitored using the sensor well 620, and a user can determine what action (if any) to take with regard to the cell culture population in the bioreactor system 600 in response to information relayed from the sensing element 628.

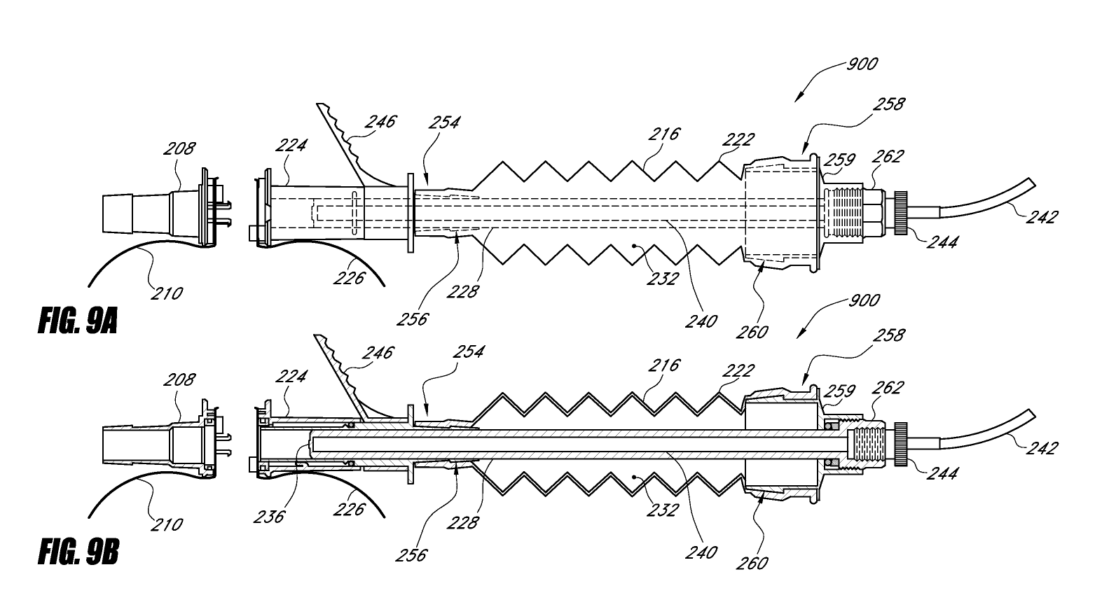

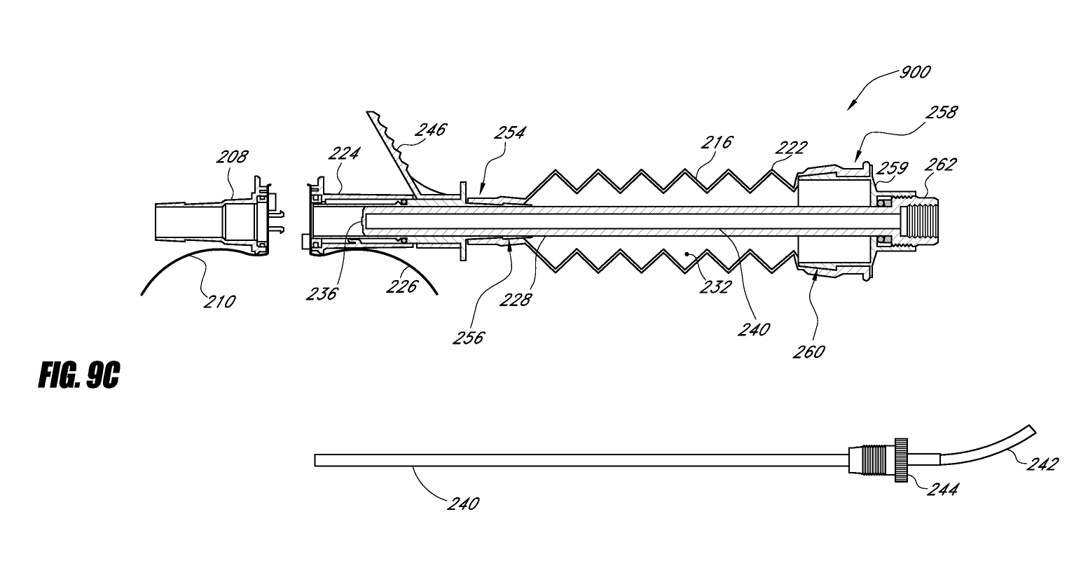

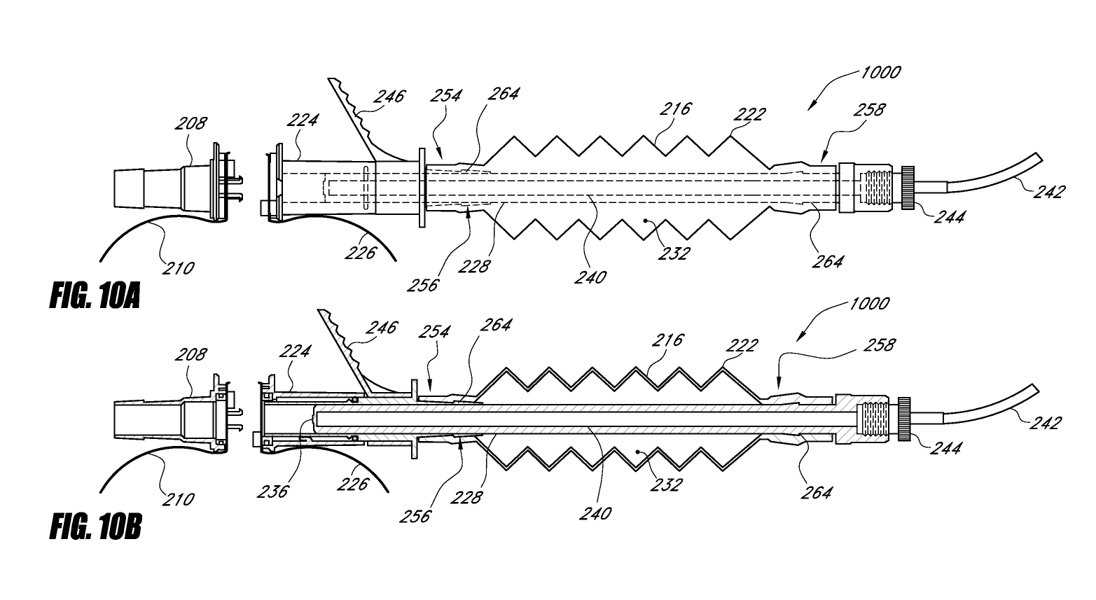

FIGS. 9a, 9b, and 9c illustrate a specific exemplary embodiment of a sensor well 900 and show certain detail and structure in greater detail. This embodiment contains some parts similar to those described above and are thus similarly labeled. These figures depict a sensor well 900 comprising an enclosure 216 with bellows 222, a connector 224, a sheath 228, a sensing element 236, and a signal transmitter 240. The connector 224 can be attached to the enclosure 216 at the distal joint end 254 of the enclosure 216. The proximal joint end 256 of the connector 224 can be inserted into the distal joint end 254 of the enclosure 216, sealably connecting the two components. At the proximal joint end 258 of the enclosure 216 a threaded insert 259 can be sealably attached to the enclosure 216 by inserting the distal joint end 260 of the threaded insert 259 into the proximal joint end 258 of the enclosure 216. A locking nut 262 which can be attached to the sheath 228 can then be screwed into the threaded insert 259 attaching the sheath 228 to the enclosure 216. As depicted in the figures, the signal transmitter 240 can be inserted into the sheath 228 and secured by a support 244 which can be threaded into the sheath 228. Each of these joints (or couplings), including but not limited to distal joint end 254 and proximal joint end 258 of the enclosure 216, proximal joint end 256 of the connector 224, and distal joint end 260 of the threaded insert 259, may be further secured through the use of clamps, latches, adhesives, or any other known securement means.

The signal transmitter 240 is configured to provide signals to and/or receive signals from the sensing element 236 and provide signals to and/or receive signals from a sensor controller, or processor (not shown). In one embodiment, the signal transmitter 240 is an optical waveguide. In another embodiment, the signal transmitter 240 is a communication line or wire. In yet another embodiment, a removable fiber optic assembly includes a signal transmitter 240, a support 244, and a communication line 242. The removable fiber optic assembly can be inserted into the sheath 228. In FIG. 9b, for example, the signal transmitter 240 is shown inserted into the sheath 228. In FIG. 9c, the signal transmitter 240 is shown removed from the sheath 228.

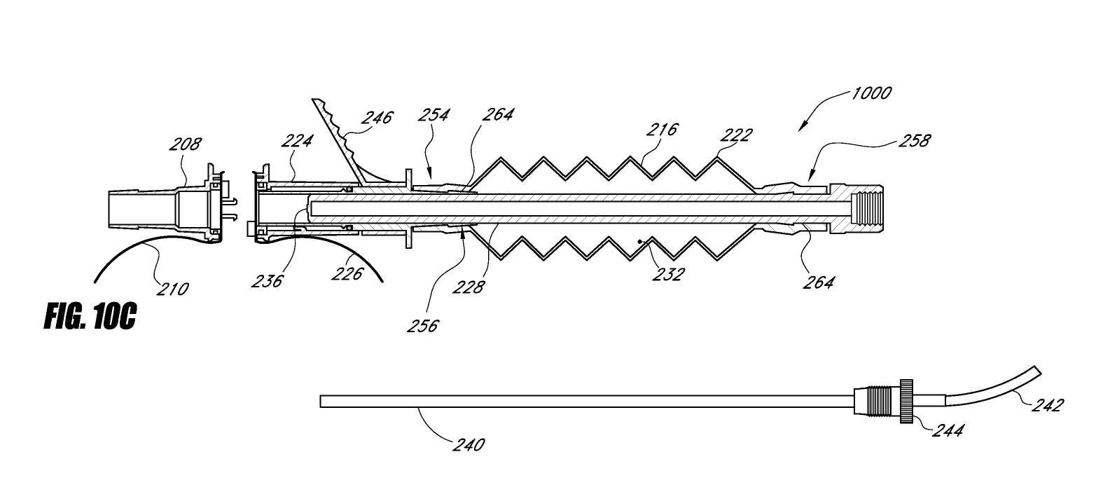

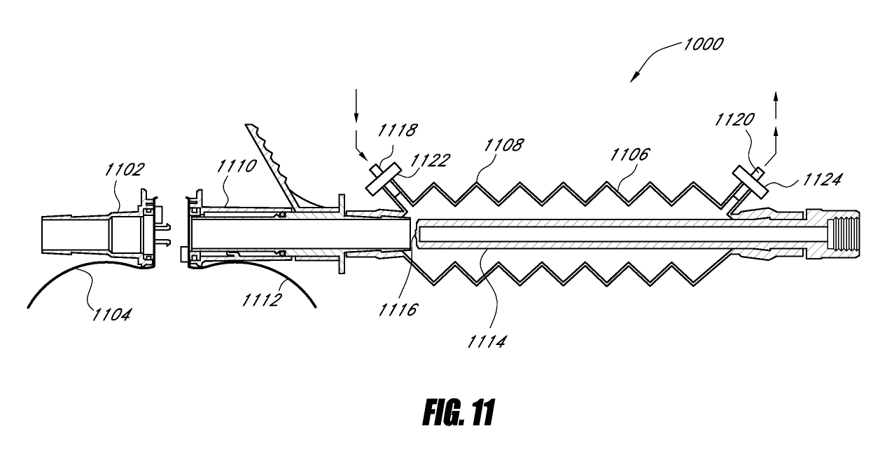

FIGS. 10a, 10b, and 10c illustrate exemplary embodiment of another sensor well 1000. These figures contain some parts similar to those described above and are thus similarly labeled. These figures depict a sensor well 1000 comprising an enclosure 216 with bellows 222, a connector 224, a sheath 228, a sensing element 236, and a signal transmitter 240. The connector 224 can be attached to the enclosure 216 at the distal joint end 254 of the enclosure. The proximal joint end 256 of the connector 224 can be inserted into the distal joint end 254 of the enclosure 216 connecting the two components. This connection may be secured and sealed by heat fusing 264 the distal joint end 254 of the enclosure 216 to the proximal joint end 256 of the connector 224. At the proximal joint end 258 of the enclosure 216 a sheath 228 can be sealably attached to the enclosure 216 by inserting the sheath 228 into the proximal joint end 258 of the enclosure 216. This joint can be secured and sealed by heat fusing 264 the sheath 228 to the proximal joint end 258 of the enclosure 216. As depicted in the figures, the signal transmitter 240 can be inserted into the sheath 228 and secured by a support 244 which can be threaded into the sheath 228.

As described above with reference to FIGS. 9a-9c, the signal transmitter 240 is configured to provide signals to and/or receive signals from the sensing element 236 and provide signals to and/or receive signals from a sensor controller, or processor (not shown). In one embodiment, the signal transmitter 240 is an optical waveguide. In another embodiment, the signal transmitter 240 is a communication line or wire. In one example, the signal transmitter 240 is a wire that provides a signal to an optical element that irradiates the sensing element, and/or receives a signal from a detector that receives illumination from the sensing element 236. In yet another embodiment, a removable fiber optic assembly includes a signal transmitter 240, a support 244, and a communication line 242. The removable fiber optic assembly can be inserted into the sheath 228. In FIG. 10b, for example, the signal transmitter 240 is shown inserted into the sheath 228. In FIG. 10c, the signal transmitter 240 is shown removed from the sheath 228.

FIG. 11 illustrates another exemplary embodiment of a sensor well 1100. The sensor well 1100 in this embodiment can be attached to a sterile connector 1102 which can have a removable seal 1104 and can be incorporated into a bioreactor system. The sensor well 1100 comprises a sterilizable enclosure 1106 which may have bellows 1108, a sterile connector 1110 which can have a removable seal 1112, a sheath 1114 which can be attached to the enclosure 1106, and a sensing element 1116 which can be located on the tip of the sheath 1114. This embodiment further comprises an inlet port, or calibration gas inlet 1118, an outlet port, or calibration gas outlet 1120, and a first 1122 and second 1124 submicron filter. The inlet and outlet ports 1118, 1120 can allow the passage of a sterile gas through the sealed enclosure 1106. This gas can be cleansed by the first 1122 and second 1124 submicron filters. This gas can be used to zero and/or calibrate the sensing element 1116 or to verify the calibration data for the sensing element 1116 without disturbing the aseptic condition of the enclosure 1106. The inlet 1118 and outlet 1120 ports can be formed as part of the enclosure 1106, or can be formed as separate components and then attached to the sensor well 1100.

Although illustrated within the context of a disposable bioreactor system, embodiments of the present invention may also be used with other containers and systems for which an ability to monitor media contained therein, while maintaining a sterile environment therein, is desirable. In addition, it will be understood by those of skill in the art that numerous and various modifications can be made without departing from the spirit of the present invention. Therefore, it should be clearly understood that the forms of the invention described herein are illustrative only and are not intended to limit the scope of the invention. In addition, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others.

What is claimed is:

1. A sensor well for use with a bioreactor vessel, the sensor well comprising:
a sheath having a proximal end and a distal end, wherein the sheath is enclosed at the distal end;
a sensing element comprising a fluorescing material disposed on the distal end of the sheath;
a signal transmitter comprising an optical waveguide disposed within at least a portion of the sheath, one end of the signal transmitter located at the distal end of the sheath, the signal transmitter positioned to transmit light from a sensor controller to illuminate the sensing element, the optical waveguide configured to receive illumination signals from the sensing element and provide signals from the sensing element to the sensor controller;
a connector comprising an aperture, the connector configured to attach the sensor well to a portion of a bioreactor vessel; and
a collapsible bellows coupled on one end to the connector and on the other end to the proximal end of the sheath, the bellows housing the sheath when the sheath is in an undeployed position not extending through the aperture in the connector, wherein the bellows, the connector, and the sheath are connected to form at least a portion of a hermetically sealable and sterilizable enclosure,
wherein the sheath and the bellows are configured so that the sheath can be deployed through the aperture in the connector and into a bioreactor vessel that is attached to the connector thereby exposing the sensing element on the distal end of the sheath to media contained in the bioreactor vessel while maintaining a sterile environment in the bioreactor vessel.

2. The sensor well of claim 1, wherein the fluorescing material is configured as a fluorescent dot.

3. The sensor well of claim 1, further comprising a plurality of sensing elements and a plurality of signal transmitters, each signal transmitter associated with at least one sensing element.

4. The sensor well of claim 1, wherein the sensing element further comprises an electronic sensor.

5. The sensor well of claim 1, wherein the bellows comprises a telescoping structure.

6. The sensor well of claim 1, wherein the bellows comprises a flexible material configured in an accordion-like structure.

7. The sensor well of claim 1, wherein the fluorescing material is configured to indicate pH.

8. The sensor well of claim 1, wherein the fluorescing material is configured to indicate dissolved oxygen content.

9. The sensor well of claim 1, wherein the fluorescing material is configured to indicate carbon dioxide content.

10. The sensor well of claim 1, wherein the proximal end of the sheath comprises an aperture configured to accept the signal transmitter.

11. A device for use with a bioreactor system, the device comprising:
a waveguide;
a sheath surrounding the waveguide;
a sensing element comprising a fluorescing material disposed at a distal end of the sheath, the sheath and sensing element configured to move between a first position and a second position;
a sterilizable enclosure configured to protect the sensing element from an exterior environment at least when the sheath and the sensing element are in the first position; and
a sterile connector disposed at a distal end of the enclosure, wherein the distal end of the sheath is disposed distal of the sterile connector when the sheath and the sensing element are in the second position,
wherein the waveguide is positioned to transmit light from a sensor controller to illuminate the sensing element, the waveguide further configured to receive illumination signals from the sensing element and provide signals from the sensing element to the sensor controller.

12. The device of claim 11, wherein the sensing element comprises one or more dots each comprising fluorescing material.

13. The device of claim 12, wherein at least one of the dots is configured to fluoresce, when radiated, to indicate a characteristic of a cell culture population to which it is exposed.

14. The device of claim 12, wherein one or more dots are configured to fluoresce to indicate pH.

15. The device of claim 12, wherein one or more dots are configured to fluoresce to indicate dissolved oxygen content.

16. The device of claim 12, wherein one or more dots are configured to fluoresce to indicate carbon dioxide content.

17. The device of claim 11, wherein the sensing element further comprises an electronic sensor.

18. The device of claim 17, wherein the sensing element is configured to measure temperature.

19. The device of claim 17, wherein the sensing element is configured to measure conductivity.

20. The device of claim 17, wherein the sensing element is configured to measure osmolality.

21. A device for use with a bioreactor vessel, the device comprising:
a sensor configured to move between a first position and a second position, the sensor comprising
a signal transmitter comprising an optical waveguide,
a sheath at least partially surrounding the signal transmitter,
and at least one sensing element comprising fluorescing material and disposed at a distal end of the sheath;
bellows surrounding at least a portion of the sensor at least when the sensor is in the first position; and
a sterile connector disposed at a distal end of the bellows, wherein the bellows, the sterile connector, and the sheath form a sterilizable enclosure when the sensor is in the first position, and wherein the sensor is movable with respect to the connector such that the distal end of the sheath is disposed distal of the sterile connector when the sensor is in the second position,
wherein the optical waveguide is positioned to transmit light from a sensor controller to illuminate the sensing element, the waveguide further configured to receive illumination signals from the sensing element and provide signals from the sensing element to the sensor controller.

22. The device of claim 21, wherein the sensing element comprises a fluorescent dot.

23. The device of claim 21, wherein the signal transmitter further comprises a communication wire.

24. A device for use with a bioreactor vessel, the device comprising:
a sensor comprising at least one waveguide and at least one sensing element comprising a fluorescing material; and
an enclosure defining a sterilizable space around the sensor, the enclosure comprising a connector configured to provide a sterile connection between the enclosure and a corresponding connector on a bioreactor vessel,
wherein the at least one waveguide is positioned to transmit light from a sensor controller to illuminate the at least one sensing element, the at least one waveguide further configured to receive illumination signals from the at least one sensing element and provide signals from the at least one sensing element to the sensor controller.

25. A device for monitoring media in a bioreactor bag having at least one port with a first sterile connector coupled thereto, the device comprising:
means for monitoring a characteristic of the media, the monitoring means comprising at least one fluorescent dot, means for transmitting light from a sensor controller to the fluorescent dot, and means for transmitting a response signal from the fluorescent dot to the controller;
means for protecting a sterile environment of the monitoring means;
means for making a sterile connection between the monitoring means and the first sterile connector; and
means for inserting the monitoring means into the bioreactor bag once a sterile connection has been made.

26. The device of claim 25, wherein the means for transmitting light comprises an optical waveguide.

27. The device of claim 25, wherein the means for transmitting the response signal from the fluorescent dot to the controller comprises an optical waveguide.

28. A bioreactor system comprising:
a disposable bioreactor bag comprising at least one port having a first sterile connector coupled thereto;
a sensor comprising at least one waveguide, at least one sensing element comprising a fluorescing material, and a second sterile connector, the at least one waveguide positioned to transmit light from a sensor controller to illuminate the at least one sensing element, the at least one waveguide further configured to receive illumination signals from the at least one sensing element and provide signals from the at least one sensing element to the sensor controller; and
an enclosure defining a sterilizable space around at least the at least one sensing element, the enclosure comprising a connector configured to provide a sterile connection between the enclosure and a corresponding connector on a bioreactor bag.

29. A method of monitoring media in a bioreactor vessel, the method comprising:
coupling a first sterile connector of the bioreactor vessel to a second sterile connector of a disposable sensor well, the sensor well comprising bellows and an injectable member, the bellows, the injectable member, a seal of the second sterile connector, and the second sterile connector forming a sterilizable space therebetween, the injectable member comprising at least one waveguide disposed inside the injectable member and at least one fluorescent dot on an end of the injectable member, the end of the injectable member disposed in the sterilizable space before the injectable member is injected into the bioreactor vessel;
removing the seal of the second sterile connector;
injecting the injectable member into the bioreactor vessel to expose the fluorescent dot to the inside of the bioreactor vessel;
transmitting, through the at least one waveguide, a particular wavelength of light from a sensor controller to illuminate the at least one fluorescent dot;
receiving illumination signals at the at least one waveguide from the at least one fluorescent dot; and
providing signals from the at least one fluorescent dot to the sensor controller using the at least one waveguide.
